(19) United States
(12) Patent Application Publication
(10) Pub. No.: US 2024/0382184 A1
(43) Pub. Date: Nov. 21, 2024

(54) SPECIMEN RECEPTACLE SYSTEM FOR AN IMAGING APPARATUS FOR IMAGING EX-VIVO TISSUE SPECIMENS

(71) Applicant: XEOS MEDICAL NV, Gent (BE)

(72) Inventors: Roel VAN HOLEN, Melsen (BE); Vincent KEEREMAN, Gent (BE); Jared W. MOORE, Gent (BE)

(21) Appl. No.: 18/576,162

(22) PCT Filed: Jun. 3, 2022
(86) PCT No.: PCT/EP2022/065241

§ 371 Date: Jan. 3, 2024

(30) Foreign Application Priority Data
Jul. 8, 2021 (EP) 21184424.6

(51) Int. Cl. A61B 10/00; G06T 7/00

(52) U.S. Cl. CPC A61B 10/0096; G06T 7/0012; G06T 2207/10081; G06T 2207/10104; G06T 2207/30204

(57) ABSTRACT

A specimen receptacle system is for an imaging apparatus for imaging ex-vivo tissue specimens. The specimen receptacle system includes a specimen receptacle having a bottom and an upstanding wall. The system further comprises an additional bottom plate which is attachable to the bottom of the specimen receptacle. The additional bottom plate is configured to receive the ex-vivo tissue specimen.

### FIELD OF THE INVENTION

The present invention generally relates to a specimen receptacle system for an imaging apparatus for imaging ex-vivo tissue specimens.

### BACKGROUND OF THE INVENTION

In the field of ex-vivo tissue specimen analysis, an important issue is the evaluation of resection accuracy. After resecting for example tumorous tissues, the surgical margins of the resected tissues need to be assessed in order to be able to decide whether more tissue has to be resected or not since tumorous tissues need to be removed as completely as possible. Thereto, the resected tissues need to have a sufficient margin of tumour free tissues. Such an evaluation of resection accuracy may be done by a histopathologist. However, the presence or stand-by of a histopathologist during an operation may be relatively expensive and may be logistically relatively difficult to achieve. At the same time, the histopathological evaluation may be a relatively time-consuming process and may be difficult to realize intraoperatively. Therefore, such a histopathological evaluation is rarely performed intraoperatively, resulting in an undesirable scheduling of additional subsequent surgeries in case of post-surgical detection of tumorous margins in the resected tissues.

It is known to use one or more imaging techniques in the evaluation of resection accuracy of ex-vivo tissue specimen, which imaging techniques can visualize tumorous tissue. A CT imaging module can for example detect differences in tissue densities in a tissue specimen and provide morphological information on the imaged tissue specimen, distinguishing tumorous tissue from healthy tissue. However, the contrast between tumorous and healthy tissue is often limited in CT images. A PET imaging module can detect the distribution of a positron-emitting radiotracer, administered to a patient before imaging, in the body of said patient. As some radiotracers are taken up in tumorous tissue with high specificity, a PET imaging module can for example provide images of tumorous tissue with a relatively high accuracy. Unfortunately, PET images may not provide detailed morphological information. Combining images made by a CT imaging module and by a PET imaging module can therefore be very advantageous in clinical imaging, in particular in margin assessment of a resected tissue specimen.

An important issue in margin assessment of a resected tissue specimen is the ex-vivo orientation of the tissue specimen with respect to a body of which the tissue has been resected. Different techniques are in use to define the orientation of the tissue specimen. The surgeon can for example indicate an anterior, posterior, superior or inferior side of the tissue specimen by using specific wires or colour paints indicative of said side or by using labelled markers or clips. An orientation of the specimen can for example be prescribed by a provider of a specimen container for an imaging apparatus. However, such a prescription is prone to human manipulation and/or handling errors and can lead to an erroneous margin assessment with respect to the original orientation of the tissue specimen in the body.

It is therefore an aim of the present invention to solve or at least alleviate one or more of the above-mentioned problems. In particular, the invention aims at providing a specimen receptacle system for an imaging apparatus for imaging ex-vivo tissue specimens which allows relative flexibility in an orientation of the resected specimen while minimizing a risk of potential handling errors.

### SUMMARY OF THE INVENTION

To this aim, according to a first aspect of the invention, there is provided a specimen receptacle system for an imaging apparatus for imaging ex-vivo tissue specimens. In particular, the specimen receptacle system comprises a specimen receptacle having a bottom and an upstanding wall. The specimen receptacle system further comprises an additional bottom plate which is attachable to the bottom of the specimen receptacle. The additional bottom plate can for example be attached to the bottom of the specimen receptacle in a permanent manner, for example by gluing or in any other suitable way. Alternatively, the additional bottom plate may be removably attachable to the bottom of the specimen receptacle. The additional bottom plate is configured to receive the ex-vivo tissue specimen in an orientation which may be chosen by the user. The additional bottom plate includes at least two distinctive orientation markers configured to indicate at least two substantially transverse anatomical directions, which are assignable to said at least two distinctive orientation markers depending on the orientation of the ex-vivo tissue specimen on the additional bottom plate chosen by the user. The at least two orientation markers can for example include at least two distinctive symbols, for example at least two among a star, a square, a disk, a polygon, a cross or whatever other symbol, as will be clear to the person skilled in the art. Said at least two orientation markers are preferably placed along a periphery of the additional bottom plate, such that said markers can remain visible when the ex-vivo tissue specimen is placed on the additional bottom plate. Contrary to prior art specimen receptacle systems, the ex-vivo tissue specimen can be placed on the additional bottom plate in an orientation chosen by the user instead of being imposed by the manufacturer of the specimen receptacle system. In determining an optimal orientation for the ex-vivo tissue specimen in the specimen container, the user can for example take into account stability of the positioning of the ex-vivo tissue specimen and/or particularities of the tissue specimen needing to be imaged. It is however preferred that the user chooses an orientation for the ex-vivo tissue specimen such that an anatomical direction of the ex-vivo tissue specimen corresponds to one of the at least two distinctive orientation markers. The choice for an orientation of the ex-vivo tissue specimen on the additional bottom plate is therefore preferably not entirely random but chosen among a number of potential orientations, the number of potential orientations depending on the number of distinctive orientation markers on the additional bottom plate. The user can then, in function of the chosen orientation of the ex-vivo tissue specimen, assign a significance, in particular an anatomical direction, to the orientation markers. In other words, the two distinctive orientation markers allow assignation of at least two substantially transverse anatomical directions to said at least two distinctive orientation markers depending on the orientation of the ex-vivo tissue specimen chosen by the user. A first symbol can be assigned a first anatomical direction or orientation, for example ‘posterior’, and a second symbol can be assigned a second anatomical direction or orientation which is substantially transverse to the first direction, for example ‘superior’ or ‘inferior’ or ‘left’ or ‘right’. By assigning at least two transverse anatomical directions, any orientation within one of the three anatomical planes, the sagittal or longitudinal plane, the transverse plane, and the coronal or frontal plane, can be defined. The assignation of at least two substantially transverse anatomical directions to said at least two distinctive orientation markers may be different at every use of the specimen receptacle. In this way, manipulation of ex-vivo tissue specimens can be simplified allowing more flexibility, and manipulation errors can be avoided, or at least be decreased.

The additional bottom plate can advantageously be made of foam. Foam can be substantially invisible to X-rays. As a result, an X-ray image of a tissue specimen on foam will give an impression of a floating tissue specimen which can simplify automated image treatment since there is a clear border of the specimen avoiding artefacts in the image. Moreover, an additional bottom plate in foam can provide several practical advantages in use for receiving ex-vivo tissue specimens. Foam can at least partly absorb body fluids of the ex-vivo tissue. Additionally, foam can allow relatively easy fixation of the ex-vivo tissue specimen on the additional bottom plate, for example by pinning the specimen into the foam or fixating the specimen in any other way known to the person skilled in the art. Foam can for example be closed cell foam, such as for example polyethylene (PE) or ethyl vinyl acetate (EVA), which is relatively robust and can for example allow writing on the foam. The foam is preferably white foam or a foam in any light colour. White or a light colour enhances visibility of colours when paint is used on the foam for markings or indicators.

The specimen receptacle can preferably include an inwardly protruding edge. The additional bottom plate may include a corresponding recess configured to receive said protruding edge of the specimen receptacle. In this way, the additional bottom plate can be inserted into the specimen receptacle in a single way only. As a result, the orientation markers on the additional bottom plate can always be oriented similarly with respect to the specimen receptacle. The inwardly protruding edge can protrude from the bottom of the specimen receptacle or from the upstanding wall of the specimen receptacle or from both the upstanding wall and the bottom of the specimen receptacle. The protruding edge and the recess can have any suitable shape as long as the shapes are corresponding shapes such that the protruding edge can engage the recess in only one way. Alternatively, the specimen receptacle may include a recess or an outwardly protruding edge, and the additional bottom plate may include a corresponding protruding edge fitting into said recess of the specimen receptacle.

The additional bottom plate can preferably include at least four distinctive orientation markers configured to indicate at least four anatomical directions of which at least two anatomical directions are substantially transverse to each other. More preferably, every two adjacent orientation markers can be configured to indicate substantially transverse anatomical directions. The at least four distinctive orientation markers can for example be assigned to four anatomical directions within one of the three anatomical planes. Even if an orientation can already be defined by two transverse directions, at least four distinctive orientation markers can help in defining an orientation of an ex-vivo tissue specimen beyond doubt, for example when the ex-vivo tissue specimen partly covers one or more of the orientation markers. Moreover, at least four distinctive orientation markers can increase a number of potential orientations for the ex-vivo tissue specimen. Even if the user can choose a most optimal orientation for the ex-vivo tissue specimen, the choice for said orientation is not entirely random. It is still preferred to position and orient the ex-vivo tissue on the additional bottom plate of the specimen receptacle in such a way that one anatomical direction of the ex-vivo tissue specimen corresponds to one of the at least two or more distinctive orientation markers. Four distinctive orientation markers, of which every two adjacent orientation markers are configured to indicate substantially transverse anatomical directions, can for example provide 24 preferred potential orientations for the ex-vivo tissue specimen on the additional bottom plate. Every additional set of four orientation markers, of which every two adjacent orientation markers are configured to indicate substantially transverse anatomical directions, can multiply said number of preferred potential orientations for the ex-vivo tissue specimen on the additional bottom plate. Such an additional set of four orientation markers may for example be rotated over for example 30° or 45° with respect to a first set of four distinctive orientation markers. It will be understood by the person skilled in the art that the at least four orientation markers are at least four distinctive orientation markers, for example at least four different symbols. Such orientation markers may differ in shape, or in colour, or both in shape and in colour.

The specimen receptacle preferably is at least partly transparent. In particular, the upstanding wall of the specimen receptacle can be transparent. More preferably, the entire specimen receptacle is transparent. The specimen receptacle is preferably transparent to different ranges of wavelengths, in particular, to X-rays. It is preferred to have a specimen receptacle which is additionally transparent to visible light such that visual inspection of the ex-vivo tissue is possible. The specimen receptacle can for example be made of a transparent plastic, such as acrylic or styrene acrylonitrile. The specimen receptacle may for example be made by injection moulding.

It is preferred that the specimen receptacle is substantially cylindrical. Such a shape can optimize a surface area of the bottom of the specimen receptacle with respect to a surface area of the upstanding wall. Alternatively, other shapes are possible as well. The upstanding wall may preferably be high enough to provide a lateral support to the ex-vivo tissue specimen. At the same time, the upstanding wall can preferably be not too high such as not to hinder insertion of the ex-vivo tissue specimen into the receptacle. A height of the upstanding wall can for example be included in a range of more or less 3 cm to more or less 10 cm, more preferably around 4 cm to more or less 7 cm, for example around 5 cm. A diameter or analogous width of the specimen receptacle can preferably correspond to a field-of-view of an imaging apparatus in which the specimen receptacle system can be used. A diameter of the specimen receptacle may for example be comprised in a range of more or less 9 cm to more or less 12 cm, for example be around 10.5 cm. Depending on the type of ex-vivo specimens to be imaged, dimensions of the specimen receptacle may be adapted.

An upper side of the specimen receptacle may preferably be open. An open top or upper side can allow use of a top-down camera, for example in an imaging apparatus. A top-down view or preferably image of the ex-vivo specimen in the specimen receptacle can allow a joint view on both the ex-vivo tissue specimen and on the at least two orientation markers of the additional bottom plate. Such a view can allow an automated determination of the orientation of the ex-vivo tissue specimen based on a preliminary assignment of the orientation markers to an anatomical direction. Alternatively, the specimen receptacle may include an openable upper side, for example a lid, which my advantageously be transparent to X-rays and preferably also to visible light.

An upper edge of the upstanding wall may advantageously include a visual indicator such as a notch. Such a notch or any other visual indicator may help in lining up the specimen receptacle system in an imaging apparatus, in particular with respect to a rotational orientation since the specimen receptacle system may preferably be insertable in an imaging apparatus in only one orientation.

The specimen receptacle system may further comprise a platform on which the bottom of the receptacle is releasably fixable. Fixation of the specimen receptacle on such a dedicated platform can ensure a stable positioning of the specimen receptacle, and thus of the ex-vivo tissue specimen, during the procedure of imaging the ex-vivo tissue specimen. Said platform may be integrated into a dedicated imaging apparatus or may be configured to be mountable into existing imaging apparatuses, for example by retrofitting such imaging apparatuses.

The specimen receptacle and/or the platform may be configured to allow a releasable fixating of the specimen receptacle on the platform in a single orientation only. This single orientation fixation may be obtained in many different ways. As an example, one of the bottoms of the receptacle and the platform can include at least one, preferably a plurality of, outwardly protruding edge configured to be received in a corresponding recess in the other of the bottom of the receptacle and the platform. A bottom of the specimen receptacle, in particular a periphery of the bottom, can for example include outwardly, preferably downwardly, protruding edges. To improve stability of the specimen receptacle when not fixated on the platform, it is preferred to include at least three edges, for example four edges, which can be like supporting edges on which the specimen receptacle can stand. The platform can then include corresponding recesses configured to receive the edges protruding from the bottom of the specimen receptacle. Alternatively, the bottom of the receptacle can include at least one recess configured to receive an edge upwardly protruding from the platform. In both cases, the protruding edges and corresponding recesses can only allow one way of fixating the specimen receptacle on the platform such that an orientation of the orientation markers with respect to the platform is known and always the same.

Said at least one outwardly protruding edge, either downwardly protruding from the specimen receptacle or upwardly protruding from the platform, can advantageously include a bevelled edge. A bevelled edge can guide insertion of said at least one edge into a corresponding recess, thus facilitating the manipulating of the specimen receptacle. Ends or sides or both of said at least one edge may be bevelled.

According to a further aspect of the invention, there is provided an imaging apparatus system for imaging ex-vivo tissue specimens including a specimen receptacle system the imaging apparatus. Such an image apparatus can provide one or more of the above-mentioned advantages. The image apparatus can for example include a positron emission tomography (PET) imaging module and/or a computed tomography (CT) imaging module. In a very advantageous embodiment, the imaging apparatus may be a single device comprising both a PET imaging module and a CT imaging module. The specimen receptacle system may be configured to receive the ex-vivo tissue specimen for imaging by both the CT imaging module and the PET imaging module.

According to a further aspect of the invention, there is provided a computer-implemented method, a controller and a computer program product for assigning an orientation to an ex-vivo tissue specimen. Such a method, controller and computer program product can provide one or more of the above-mentioned advantages.

### BRIEF DESCRIPTION OF THE DRAWINGS

FIG. 1 shows a perspective view on a preferred embodiment of a specimen receptacle system for an imaging apparatus for imaging ex-vivo tissue specimens according to a first aspect of the invention;

FIG. 2 shows a top view on the specimen receptacle system of FIG. 1;

FIGS. 3a and 3b show a perspective view on a further preferred embodiment of a specimen receptacle (FIG. 3b) for an imaging apparatus for imaging ex-vivo tissue specimens according to a first aspect of the invention and on the additional bottom plate (FIG. 3a) of said specimen receptacle of FIG. 3b;

FIG. 4 shows a frontal view on the specimen receptacle system of FIG. 1;

FIG. 5 shows a side view on the specimen receptacle system of FIG. 1.

FIG. 6 shows a perspective view on a preferred embodiment of a platform of the specimen receptacle system of FIG. 1;

FIG. 7 shows a perspective view on the specimen receptacle system of FIG. 1 including the platform of FIG. 6.

### DETAILED DESCRIPTION OF EMBODIMENT(S)

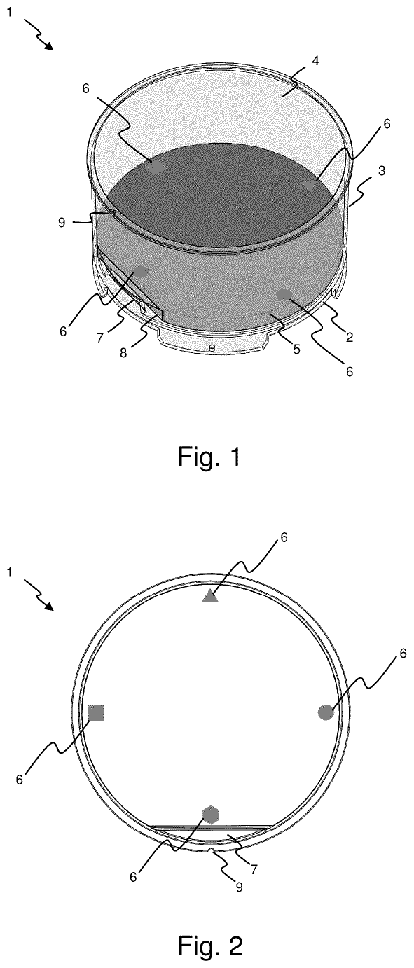

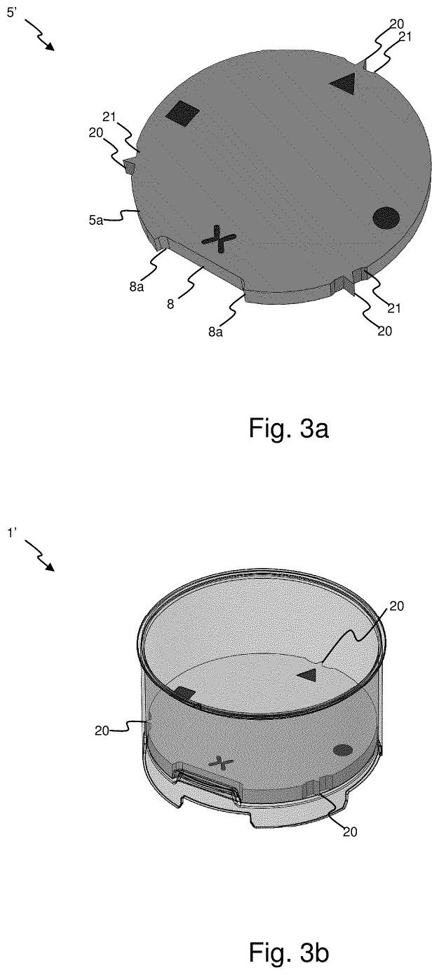

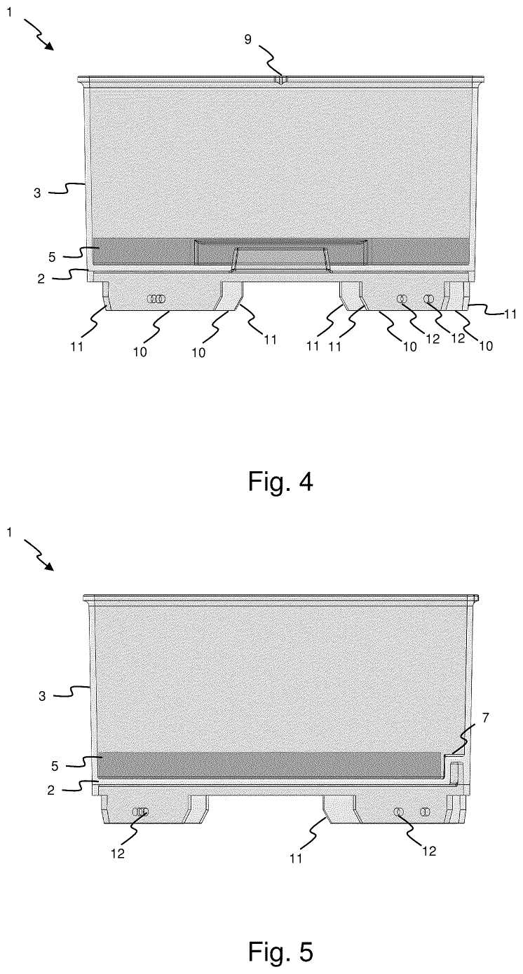

FIG. 1 shows a perspective view on a preferred embodiment of a specimen receptacle system for an imaging apparatus for imaging ex-vivo tissue specimens according to a first aspect of the invention. The specimen receptacle system comprises a specimen receptacle 1 having a bottom 2 (as seen in FIGS. 3 and 4) and an upstanding wall 3. The specimen receptacle 1 can for example be substantially cylindrical, i.e. having an upstanding wall 3 with a substantially circular cross-section. An upper side 4 of the specimen receptacle 1 is preferably open. The specimen receptacle 1 is at least partly transparent. The specimen receptacle 1 can for example be made of acrylic or styrene acrylonitrile. The specimen receptacle 1 can preferably be made by injection moulding. The system further comprises an additional bottom plate 5 which is attachable to the bottom 2 of the specimen receptacle 1. The additional bottom plate 5 can preferably be made of foam. The additional bottom plate 5 may for example be glued onto the bottom 2 of the specimen receptacle 1. The bottom plate may also be releasably attached in any other known way or rest on the bottom 2 without being attached. The additional bottom plate 5 is configured to receive the ex-vivo tissue specimen, which may for example be tumorous tissue which has just been excised during surgery. The specimen receptacle 1 can include an inwardly protruding edge 7. The inwardly protruding edge 7 may be comprised in the upstanding wall 3 of the specimen receptacle 1 or in the bottom 2, or in both, as is the case in the present embodiment. The inwardly protruding edge 7 may for example be an inwardly protruding ridge extending both from the bottom 2 as from the upstanding wall 3, as can be seen in FIG. 4. The inwardly protruding edge 7 may, but preferably does not, extend over an entire height of the upstanding wall 3, in order not to compromise a volume of the specimen receptacle 1. A height of said inwardly protruding edge 7 may for example correspond to a height of the additional bottom plate 5, as can be seen in FIG. 4. As such, the additional bottom plate 5 may be substantially flush with an upper side of the inwardly protruding edge or ridge 7. The shape of the inwardly protruding edge 7 can vary. Here, the inwardly protruding edge 7 substantially extends along a chord of the cross-section of the specimen receptacle 1 such that said cross-section of the specimen receptacle 1 at a level of the inwardly protruding edge 7 diverges from a substantially circular cross-section. The additional bottom plate 5 can then advantageously include a corresponding recess 8 configured to receive said protruding edge of the specimen receptacle. A shape of the recess 8 preferably corresponds to a shape of the protruding edge 7. In the present embodiment, the additional bottom plate 5 is substantially disk-shaped missing however a substantially circular segment, being the recess 8. An upper edge of the upstanding wall 3 can include a notch 9, for example just one notch, for example located substantially above the inwardly protruding edge 7. Such a notch or any other visual indicator can help and guide a user to correctly orientate the specimen receptacle system and facilitate insertion of the specimen receptacle system into an imaging system.

FIG. 2 shows a top view on the specimen receptacle system of FIG. 1. The additional bottom plate 5 includes at least two distinctive orientation markers 6 configured to indicate at least two substantially transverse anatomical directions, in particular to allow assignation of at least two substantially transverse anatomical directions to said at least two distinctive orientation markers 6. More preferably, the additional bottom plate 5 can include at least four distinctive orientation markers 6, for example a square, a triangle, a disk and a hexagon as shown in FIGS. 1 and 2, or for example a square, a triangle, a disk and a cross as shown in FIGS. 3a, 3b and 7. Other types of symbols can also be used as orientation markers 6. Said distinctive orientation markers are preferably located eccentrically, for example towards and/or along a perimeter of the additional bottom plate 5 rather than towards a middle region of said additional bottom plate 5 so that these orientation markers 6 can remain visible when the ex-vivo tissue specimen is positioned in an orientation chosen by the user on the additional bottom plate 5. The orientation markers 6 are positioned such that every two adjacent orientation markers 6 are configured to indicate substantially transverse anatomical directions. There are three independent anatomical axes which always have the same orientation relative to each other. Each of the three independent axes has two labels to represent the two directions along the axis. Anatomical directions may for example include superior, inferior, anterior, posterior, left, right, medial, lateral or any other as known to the person skilled in the art depending on the anatomical plane used. Different medical specialties may have different preferences for the labelling of anatomical directions. The user, for example a surgeon or a nurse, can first position an ex-vivo tissue specimen into the specimen receptacle 1 in an orientation which the user believes to be a most optimal orientation and can in a next step assign an anatomical direction to at least one of said distinctive orientation markers 6. The opposite anatomical direction can then automatically be deduced. As an example, the triangle may be assigned to indicate a posterior direction. The opposite orientation marker, if any, in particular the hexagon, may then be automatically assigned to the anterior direction by deduction. In a next step, the user is left with four remaining anatomical directions and at least one orientation marker, or two opposite orientation markers. The user may then assign one of said four remaining anatomical directions, for example a left direction, to the disk or to the square. The opposite anatomical direction can then be automatically assigned to the opposite orientation marker. In that example, the plane of the additional bottom plate 5 can for example correspond to a transverse plane of the body such that the square orientation marker will correspond to a right direction and the hexagon orientation marker can indicate an anterior direction. The directions indicated by the orientation markers 6 can be redefined at every use of the specimen receptacle. This way of proceeding is contrary to what is done in the prior art, where an orientation of the ex-vivo tissue specimen is generally imposed by the manufacturer of the specimen receptacle. In the present invention, the user is allowed considerably more freedom in positioning the ex-vivo tissue specimen into the receptacle 1 such that the user can choose a relatively stable way of positioning the specimen which is advantageous for imaging. The user only needs to assign anatomical directions to the distinctive orientation markers 6 provided on the additional bottom plate 5 of the specimen receptacle 1.

FIG. 3b shows a perspective view on a further preferred embodiment of a specimen receptacle 1′ for an imaging apparatus for imaging ex-vivo tissue specimens according to a first aspect of the invention and FIG. 3a shows a perspective view on the additional bottom plate 5′ of said specimen receptacle 1′ of FIG. 3b. The additional bottom plate 5′ differs from the embodiment shown in FIGS. 1 and 2 in that the additional bottom plate 5′, in particular a circumference 5a of the additional bottom plate 5′, includes at least one, and preferably a plurality of radially extending protrusions 20 or tabs. The circumference 5a may further include at least one, and preferably a plurality of recesses 21 from which said at least one protrusion 20 extends. The at least one protrusion 20 is preferably made of a flexible material such that the at least one protrusion 20 is retractable into an inner side of the specimen receptacle 1′ despite its extension beyond an inner diameter of the specimen receptacle 1′. The at least one protrusion 20 can in fact be squeezed into the specimen receptacle 1′, as is shown in FIG. 3b. Said at least one recess 21 may provide room for receiving the retracted protrusion 20. The at least one protrusion 20 is preferably made of the same material as the additional bottom plate 5′, for example of a foam. The at least one protrusion 20 may have a substantially triangular shape having only one extending angle, which may be a relatively sharp angle. In this way, the at least one protrusion 20 may be squeezed into the specimen receptacle 1′ relatively easily and friction with an internal side of the specimen receptacle 1′ may be minimized to ease insertion of the additional bottom plate 5′. Other shapes are for the at least one protrusion 20 are possible as well. The plurality of protrusions 20 and corresponding recesses 21 may be distributed along a circumference 5a of the additional bottom plate 5′ at substantially equal distances, for example every 120° for three protrusions 20. Too many protrusions may hamper ease of use. The additional bottom plate 5′ further differs from the embodiment shown in FIGS. 1 and 2 in that the segment shaped recess 8 includes substantially rounded ends 8a. Thanks to the at least one protrusion 20, the additional bottom plate 5′ includes extra protection against a rotational shift of the additional bottom plate 5′ in the specimen receptacle 1′, in addition to the segment shaped recess 8 which can guarantee a fixed orientation of the additional bottom plate 5′ with respect to the specimen receptacle 1′ while the distinctive orientation markers 6 can allow a random orientation of a specimen on the additional bottom plate 5′.

FIG. 4 shows a frontal view on the specimen receptacle system of FIG. 1. The bottom 2 of the specimen receptacle 1 can include a plurality, for example four, of outwardly, preferably downwardly, protruding edges 10. Said edges 10 may be positioned and/or extend along a circumference or perimeter of the bottom 2 of the specimen receptacle 1. The downwardly protruding edges 10 can for example have a shape of curved blades along a perimeter of the bottom 2 of the specimen receptacle 1. Said edges 10 may be configured to be received in corresponding recesses of a platform on which the bottom 2 of the specimen receptacle 1 is releasably fixable. Said edges 10 may advantageously differ in length along a circumference of perimeter of the bottom 2 of the specimen receptacle 1. As a result, the specimen receptacle 1 can be received in said platform, more preferably in the corresponding recesses of said platform, in only one way. As such, the orientation of the specimen receptacle 1 with respect to the platform is always the same and known. The downwardly protruding edges 10 can be bevelled edges and can for example include a bevelled end 11 or even first and second bevelled ends 11. The downwardly protruding edges 10 can optionally further include at least one hole 12 configured to receive a snapping element to fixate the specimen receptacle 1 on the platform. Alternatively, the specimen receptacle 1 may be configured to rest on the platform by its own weight without any additional snapping or magnetic fitting or other holding elements.

FIG. 5 shows a side view on the specimen receptacle system of FIG. 1. A total height of the specimen receptacle may for example be included in a range of more or less 2 cm to more or less 12 cm. A height of the upstanding wall 3 can for example be included in a range of more or less 3 cm to more or less 10 cm, more preferably around 4 cm to more or less 7 cm, for example around 5 cm. A diameter of the specimen receptacle 1 can preferably correspond to a field-of-view of an imaging apparatus in which the specimen receptacle system can be used. A diameter of the specimen receptacle 1 may for example be comprised in a range of more or less 9 cm to more or less 12 cm, for example be around 10.5 cm.

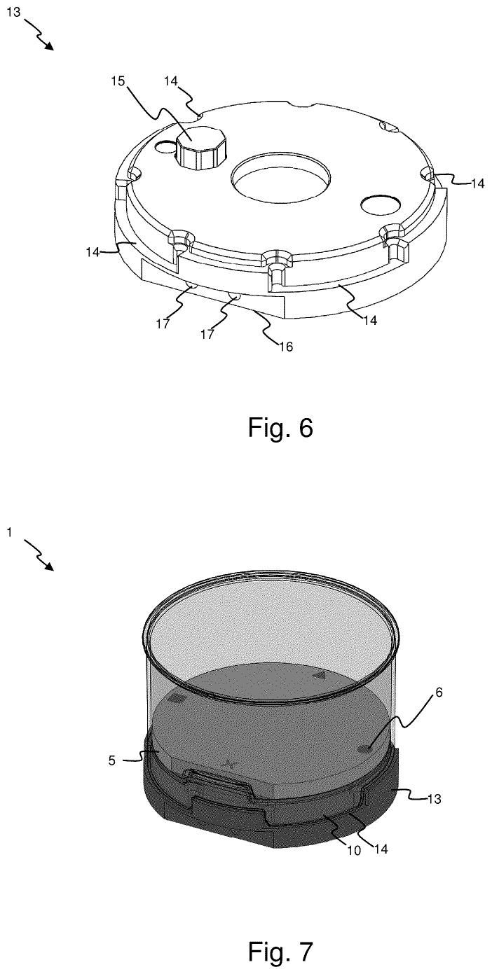

FIG. 6 shows a perspective view on a preferred embodiment of a platform 13 of the specimen receptacle system of FIG. 1 and FIG. 7 shows a perspective view on the specimen receptacle system of FIG. 1 including the platform of FIG. 6. The platform 13 may be included in an imaging apparatus or may be retrofitted into an existing imaging apparatus. The platform 13 is configured to receive the bottom of the specimen receptacle 1 in a releasably fixable way. The specimen receptacle 1 may for example be fixable by form-fitting of the specimen receptacle 1. Alternatively, the specimen receptacle 1 may be snapped onto the platform or may be held by magnetic elements or in any other way known to the person skilled in the art. The specimen receptacle 1 and/or the platform 13 are preferably configured such as to allow a releasable fixating of the specimen receptacle 1 on the platform 13 in a single orientation only. Thereto, the platform 13, preferably an outer circumference of the platform 13, may include recesses 14 which are configured to receive the outwardly, for example downwardly, extending edges 10 of the specimen receptacle 1. Said recesses 14 can for example each have a different circumferential length, such that each recess is configured to receive only one of the pluralities of downwardly extending edges 10 of the specimen receptacle 1. The platform 13 can further include a sensing button 15 which is configured to sense whether or not the specimen receptacle 13 is well positioned on the platform 13. The sensing button 15 may include a pre-tensioned element (not shown), for example a spring element, which is configured to keep the sensing button 15 in an extended position as shown, in which the sensing button 15 extends above the platform 13. Only when the specimen receptacle 1 is well positioned on the platform 13 can the sensing button be pressed into the platform 13 against the force of the pre-tensioned element. The sensing button 15 may be configured to transmit a signal to a central computing unit and/or to an imaging apparatus signalling that the specimen receptacle 1 has been correctly positioned. The platform 13, in particular a bottom side of the platform 13, may further include an additional recess 16 substantially extending along a chord of the circumference of the platform 13, the additional recess being configured to assure a correct placement of the platform 13 into an imaging apparatus, which is easier to obtain with a relatively straight and/or flat surface, such as along a chord, than along a curved surface. The additional recess 16 may include holes 17 to allow a fixed attachment of the platform 13 to an imaging apparatus, for example by screws, or by any other known attachment means.

In summary, the platform 13 is shaped such that it has a fixed orientation with respect to an imaging apparatus system. The platform 13 and the specimen receptacle are configured to allow only one orientation of the receptacle 1 with respect to the platform 13. The additional bottom plate 5 including the distinctive orientation markers 6 can only be positioned in the specimen receptacle 1 in a single way. The position of the orientation markers 6 can thus be known to the imaging apparatus system and is relatively error-proof. At the same time, the user has a relative freedom in the positioning of the ex-vivo tissue specimen into the specimen receptacle. The user can then be prompted, for example by dedicated software, to assign one of the six anatomical directions to a first of the orientation markers 6. The opposite anatomical direction can then be deduced, which leaves the user to only assign one of the four remaining anatomical directions to a second orientation marker which is on a transverse axis with respect to the first orientation marker. In this way, the other anatomical orientations can be deduced and used by the imaging apparatus system. The present invention can thus provide a specimen receptacle system for an imaging apparatus for imaging ex-vivo tissue specimens which allows relative flexibility in a positioning of the resected specimen while minimizing a risk of potential handling errors.

During surgery, for example during breast surgery, an imaging apparatus, for example a mobile imaging apparatus, may be used to perform ex-vivo tissue specimen imaging. Thereto, a surgeon, or more preferably a nurse, may move the imaging apparatus near the operating table. The surgeon or the nurse may put the excised tissue specimen in the tissue specimen receiving element, such as for example in the present specimen receptacle system in a random orientation. The surgeon, nurse or other operator can then assign an anatomical direction to at least two transverse, and preferably to four of the orientation markers 6 in function of how the tissue specimen is positioned, for example by using dedicated software thereto. The tissue specimen receiving element can then be provided to the imaging apparatus. The imaging apparatus may be shaped such as to correctly position the tissue specimen receiving element in the apparatus. An optical camera, which may be mounted substantially above the tissue specimen receiving element, for example the specimen receptacle system, may now make top view images of the tissue specimen, which may contribute to an automated determination of an orientation of the ex-vivo tissue specimen in the specimen receptacle system. Then the tissue specimen receiving element may be moved to a CT imaging module of the imaging apparatus. The CT imaging module can then perform imaging of the ex-vivo tissue specimen. In a combined PET-CT scanning apparatus, the same tissue specimen receiving element may be moved to a PET imaging module. Then the PET imaging module can perform imaging of the ex-vivo tissue specimen. An image reconstruction module can perform image reconstruction of the CT images and/or of the PET images. Reconstructed 3D PET and/or CT images can then be displayed separately and/or simultaneously on a display for evaluation by the surgeon and/or nurse or by any other medical practitioner, for high precision margin assessment of the ex-vivo tissue specimen, which can preferably be performed intraoperatively.

Although the present invention has been illustrated by reference to specific embodiments, it will be apparent to those skilled in the art that the invention is not limited to the details of the foregoing illustrative embodiments, and that the present invention may be embodied with various changes and modifications without departing from the scope thereof. The present embodiments are therefore to be considered in all respects as illustrative and not restrictive, the scope of the invention being indicated by the appended claims rather than by the foregoing description, and all changes which come within the meaning and range of equivalency of the claims are therefore intended to be embraced therein. In other words, it is contemplated to cover any and all modifications, variations or equivalents that fall within the scope of the basic underlying principles and whose essential attributes are claimed in this patent application. It will furthermore be understood by the reader of this patent application that the words “comprising” or “comprise” do not exclude other elements or steps, that the words “a” or “an” do not exclude a plurality, and that a single element, such as a computer system, a processor, or another integrated unit may fulfil the functions of several means recited in the claims. Any reference signs in the claims shall not be construed as limiting the respective claims concerned. The terms “first”, “second”, third”, “a”, “b”, “c”, and the like, when used in the description or in the claims are introduced to distinguish between similar elements or steps and are not necessarily describing a sequential or chronological order. Similarly, the terms “top”, “bottom”, “over”, “under”, and the like are introduced for descriptive purposes and not necessarily to denote relative positions. It is to be understood that the terms so used are interchangeable under appropriate circumstances and embodiments of the invention are capable of operating according to the present invention in other sequences, or in orientations different from the one(s) described or illustrated above.

1.-15. (canceled)

16. A specimen receptacle system for an imaging apparatus for imaging ex-vivo tissue specimens, the specimen receptacle system comprising a specimen receptacle having a bottom and an upstanding wall;
wherein the system further comprises an additional bottom plate which is attachable to the bottom of the specimen receptacle,
wherein the additional bottom plate is configured to receive the ex-vivo tissue specimen, and
wherein the additional bottom plate includes at least two distinctive orientation markers configured to allow assignation of at least two substantially transverse anatomical directions to said at least two distinctive orientation markers.

17. The specimen receptacle system according to claim 16, wherein the additional bottom plate is made of foam.

18. The specimen receptacle system according to claim 16, wherein the specimen receptacle includes an inwardly protruding edge, and wherein the additional bottom plate includes a corresponding recess configured to receive said protruding edge of the specimen receptacle.

19. The specimen receptacle system according to claim 16, wherein the additional bottom plate includes at least four distinctive orientation markers configured to indicate at least four anatomical directions of which at least two anatomical directions are substantially transverse to each other,
wherein in particular every two adjacent orientation markers are configured to indicate substantially transverse anatomical directions.

20. The specimen receptacle system according to claim 16, wherein the specimen receptacle is at least partly transparent.

21. The specimen receptacle system according to claim 16, wherein the specimen receptacle is substantially cylindrical.

22. The specimen receptacle system according to claim 16, wherein an upper side of the specimen receptacle is open.

23. The specimen receptacle system according to claim 16, wherein an upper edge of the upstanding wall includes a visual indicator such as a notch.

24. The specimen receptacle system according to claim 16, further comprising a platform on which the bottom of the specimen receptacle is releasably fixable.

25. The specimen receptacle system according to claim 24, wherein the specimen receptacle and/or the platform are configured to allow a releasable fixating of the specimen receptacle on the platform in a single orientation only.

26. An imaging apparatus system for imaging ex-vivo tissue specimens including a specimen receptacle system according to claim 16.

27. The imaging apparatus system according to claim 26, comprising a positron emission tomography (PET) imaging module and/or a computed tomography (CT) imaging module.

28. A computer-implemented method for assigning an orientation to an ex-vivo tissue specimen laying in a specimen receptacle according to claim 16, comprising the steps of:
optionally, displaying on a screen the at least two orientation markers and six anatomical directions;
receiving from a user input an assignation of a first orientation marker of the at least two orientation markers to a first of six anatomical directions;
optionally, displaying on a screen four of the six anatomical directions eliminating the anatomical direction to which the first orientation marker has been assigned and its opposite anatomical direction;
receiving from a user input an assignation of a second orientation marker, which is next to the first orientation marker, to a second anatomical direction which is transverse to the first anatomical direction, and which is one of the four anatomical directions displayed on the screen;
deriving from said first and second transverse anatomical directions the orientation of the ex-vivo tissue specimen;
optionally, displaying on a screen the assigned orientation of the ex-vivo tissue specimen.

29. A controller comprising at least one processor and at least one memory including computer program code, the at least one memory and computer program code configured to, with the at least one processor, cause the controller to perform the method according to claim 28.

30. A computer program product comprising computer-executable instructions for performing the method according to claim 28, when the program is run on a computer.
